(19) United States
(12) Patent Application Publication
(10) Pub. No.: US 2007/0282173 A1
(43) Pub. Date: Dec. 6, 2007

(54) Vital sign sending method and a sending apparatus thereof

(76) Inventors: Bily Wang, Hsin Chu City (TW); Chung-Hsien Su, Tainan City (TW); Yuan-Hsiu Yang, Kaohsiung City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/443,189

(22) Filed: May 31, 2006

(51) Int. Cl. A61B 5/00

(52) U.S. Cl. 600/300

(57) ABSTRACT

A vital sign sending method and a sending apparatus thereof is disclosed. The vital sign sending method includes the following steps. A user wears the vital sign sending apparatus. The vital sign sending apparatus includes a positioning module fitting with a satellite positioning system, a sending module that can transmit information via a wireless communication system, and a sensing module that contacts the user's body and monitors the vital signs of the user. Then, the vital sign sending apparatus monitors and obtains the vital signs over a fixed period and software is adopted to judge whether the vital signs of the user are abnormal or not. When the vital signs of the user are abnormal, the vital sign sending apparatus forcibly generates a sending function for informing a target person, and information regarding the location and vital signs of the user are transmitted to the target person.

### BACKGROUND OF THE INVENTION

1. Field of the Invention

The present invention relates to a vital sign sending method and a sending apparatus thereof. In particular, a vital sign sending method and a sending apparatus thereof can generate an automatic sending function under special or urgent conditions.

2. Description of the Related Art

Over time people have diligently discovered more exact navigating and locating systems, from the invention of the compass in ancient China to the first artificial satellite launched by the Soviet Union in 1957. More recently, a John Hopkins College laboratory found that tracking parameters of a satellite could be obtained via the Doppler effect of artificial satellite radio signals. Thereby, the location of people on earth could be calculated. This positioning system has developed rapidly and is extensively applied as a navigation system. The global positioning system (GPS) is the most popular. As long as they have a receiving device, anyone can easily receive signals emitted from a GPS satellite anywhere and obtain data pertaining to their exact location, speed and time. GPS can be further integrated with geographic information system (GIS) to provide navigating and tracking functions.

Due to the safety or obligation, more and more families and companies are now using a personal positioning system. These often take the form of an embedded device disposed in a cell phone. According to an order of the U.S. Federal Government, from the end of the year 2005, wireless communication proprietors have to automatically lock the location of a user making a 119 call. There exist a lot of products, such as a wristwatch type personal positioning device from Wherify Wireless and a digital angel from the Applied Digital Solutions, that can track the location of a hiker in the wild, or a pet.

The wristwatch type personal positioning device is most often used for tracking the user's location, such as the elderly or a child. When an emergency occurs and the user needs to be helped, the user has to issue an emergency signal manually to send an emergency signal to an emergency center. This is achieved by pressing a specified key or calling out a specified number. If the user is in a coma, in a state of shock, or otherwise incapable of manually activating the emergency signal, the function is useless.

Meanwhile, the digital angel is a sensor that is worn by the user and can sense the user's vital sign. The signal from the digital angel is transmitted to an Internet-connected ground station via a wireless method. The real user of the digital angel is a third person that is different form the original user (the wearing user), such as nurses, parents, hospital staff or lifesavers. The third person obtains the vital sign and the location information via the digital angel delivery system via the Internet or a telephone. If this method meets the above conditions, the user can still send out an emergency signal. However, by the time the third person is made aware of the emergency condition, it may be too late to rescue the user.

### SUMMARY OF THE INVENTION

One particular aspect of the present invention is to provide a vital sign sending method and a sending apparatus thereof. The sending apparatus constantly monitors the conditions of a user's body and forcibly generates a signal under special or urgent conditions to achieve an emergency life-saving function.

The vital sign sending method of the present invention includes the following steps. A user puts on the vital sign sending apparatus. The vital sign sending apparatus includes a positioning module fitting with a satellite positioning system, a sending module that transmits information via a wireless communication system, and a sensing module that contacts the user's body so that it can monitor the user's vital signs. Then, the vital sign sending apparatus monitors and obtains vital signs during a fixed period and a software program judges whether the vital signs of the user are abnormal or not. When the vital signs of the user are abnormal, the vital sign sending apparatus forcibly generates a sending function to inform a target person of the emergency. Furthermore, the location and the vital signs are also transmitted to the target person.

The vital sign sending apparatus of the present invention includes a sending module that transmits information via wireless communication technology, a positioning module that is linked with the sending module and receives satellite signals from a satellite positioning system, and a sensing module that is linked with the sending module and contacts the user for monitoring their vital signs. The sending module includes a wireless communication unit for providing the wireless communication technology, a software unit electrically linked with the wireless communication unit for judging whether the user's vital signs are abnormal or not, a battery unit electrically linked with the wireless communication unit and providing power, and a receiver/amplifier unit electrically linked with the wireless communication unit which provides a sound input and output function. The positioning module includes a positioning system for receiving satellite signals, and a micro processing unit.

For further understanding of the invention, reference is made to the following detailed description illustrating the embodiments and examples of the invention. The description is only for illustrating the invention and is not intended to be considered limiting of the scope of the claim.

### BRIEF DESCRIPTION OF THE DRAWINGS

The drawings included herein provide a further understanding of the invention. A brief introduction of the drawings is as follows:

FIG. 1 is a schematic diagram of a flow chart of the vital sign sending method of the present invention;

FIG. 2 is a schematic diagram of a simplified structure of the vital sign sending apparatus of the present invention;

FIG. 3 is a schematic diagram of a flow chart of another embodiment of the vital sign sending method of the present invention;

FIG. 4 is a schematic diagram of the vital sign sending apparatus of the present invention;

FIG. 5 is a schematic diagram of an application of the vital sign sending apparatus of the present invention; and

FIG. 6 is a schematic diagram of a simplified system of the vital sign sending method of the present invention.

### DETAILED DESCRIPTION OF THE PREFERRED EMBODIMENTS

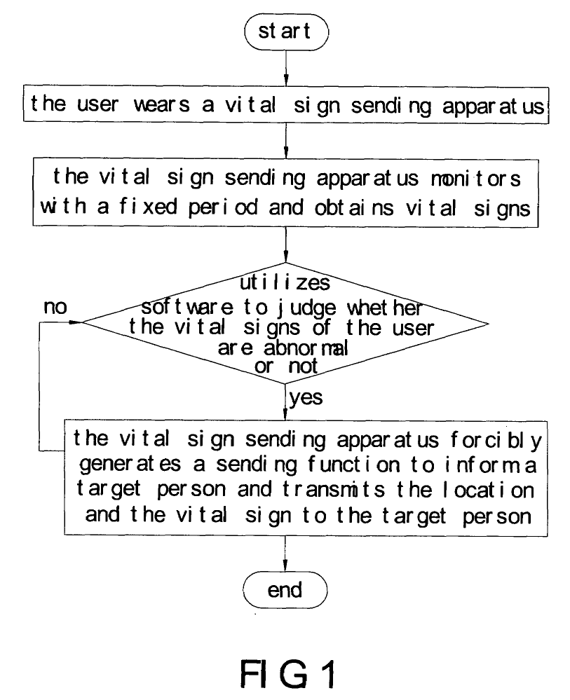

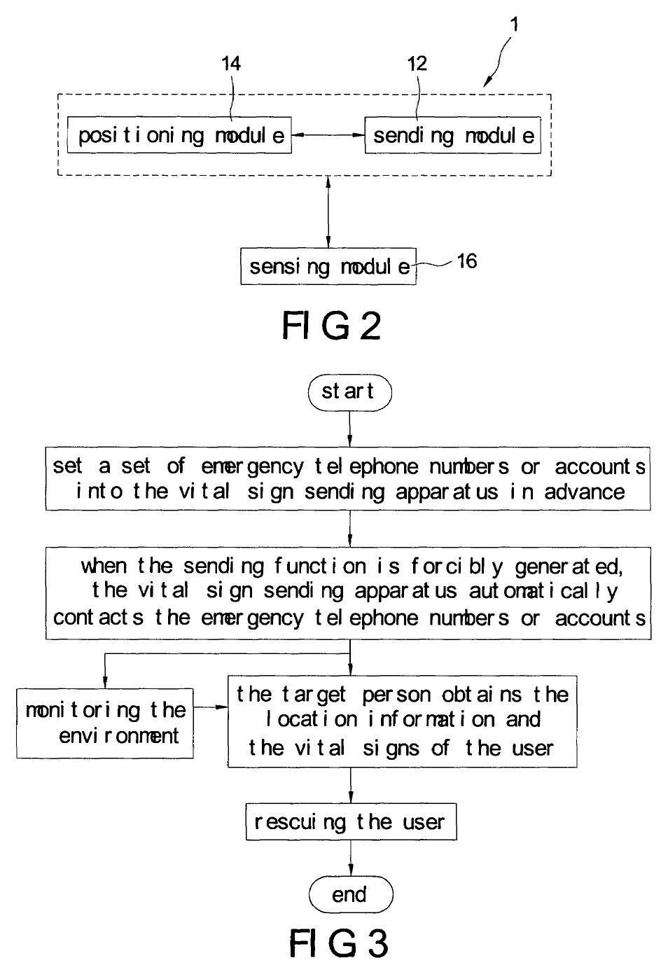

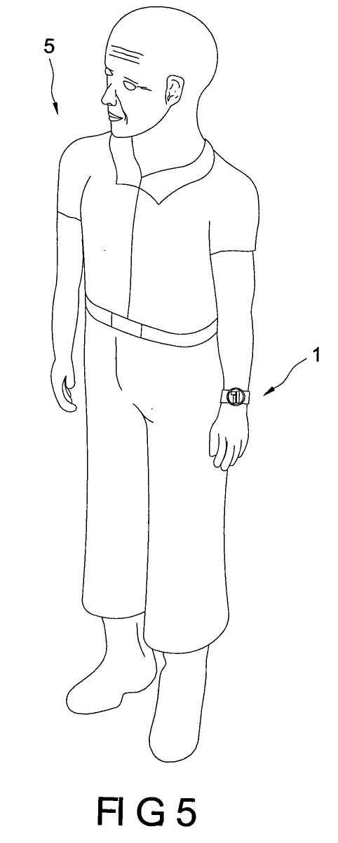

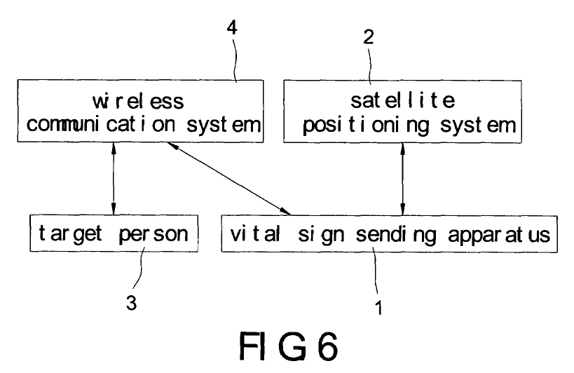

The present invention provides a vital sign sending method and a sending apparatus thereof. The sending apparatus constantly monitors the vital signs of its user and forcibly generates a sending function under special or urgent conditions to achieve the emergency lifesaving function. Please refer to FIGS. 1, 2 and 6, which show a schematic diagram of the flow chart and the apparatus of the present invention. The vital sign sending method of the present invention includes the following steps. (a) A user wears a vital sign sending apparatus 1. The vital sign sending apparatus 1 includes a positioning module 14 fitted with a satellite positioning system 2, a sending module 12 that can transmit information via a wireless communication system 4, and a sensing module 16 that contacts the user's body and monitors their vital signs. (b) The vital sign sending apparatus 1 monitors and obtains vital signs over a fixed period and utilizes a software unit 122 to judge whether the vital signs of the user are abnormal or not. Please also refer to FIG. 4. (c) When the vital signs of the user are abnormal, the vital sign sending apparatus 1 forcibly generates a sending function for informing a target person 3. (d) The location of the user and their vital signs are transmitted to the target person 3 (please refer to FIG. 6).

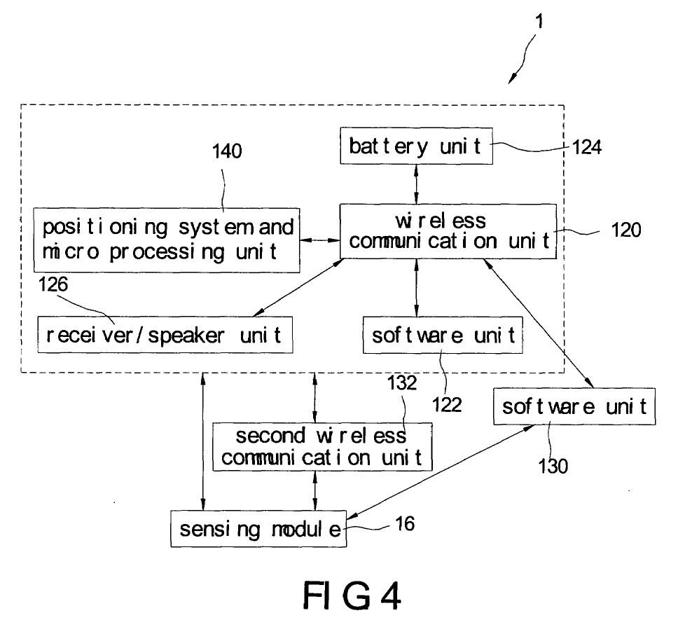

Please refer to FIG. 3. The step of a user wearing a vital sign sending apparatus 1 further comprises setting a set of emergency telephone numbers or accounts into the vital sign sending apparatus 1 in advance. Therefore, when the sending function is forcibly generated, the vital sign sending apparatus 1 can execute the automatic communication function via the emergency telephone number or account. The emergency information can be sent to the target person 3 via a variety of methods, such as making a phone call, sending a short message, or transmitting an instantaneous electronic information. The detailed methods can have the following methods. The vital sign sending apparatus 1 directly sends a short message, instantaneous electronic information or an e-mail regarding the location and vital signs of the user, to the target person 3. Alternatively, the vital sign sending apparatus 1 makes a call to the target person 3, and then guides the target person 3 via a voice system to obtain the location and vital signs of the user. When the vital sign sending apparatus 1 sends the user's location and vital signs to the target person 3, the target person 3 is informed to call the vital sign sending apparatus 1 to obtain more up-to-date information. The target person 3 can then monitor the environment of the user to decide whether another action needs to performed or not, such as informing a relative, or a nearby police station, emergency center or hospital.

The set of emergency telephone numbers or accounts are arranged in order according to their importance and used in order for automatically informing the target person 3. The target person 3 could be a person, a private organization or a governmental organization, such as a relative, a hospital, a police station, or an emergency center.

The satellite positioning system 2 can be either the global positioning system (GPS) developed by the U.S. army, the global orbiting navigation satellite system (GLONASS) developed by the Russian government, the Dipper positioning system developed by the China government, or the Galileo commercial satellite positioning system integrated with GPS and GLONASS by the governments of the European Union.

The sensing module 16 can utilize brainwaves, a pulse, a heartbeat, or sweat gland monitoring technology. The sensing module 16 senses any changes in the user's brainwaves, pulse, heartbeat or sweat glands of the user to judge whether the physiology symptoms of the user are abnormal and whether assistance needs to be given to the user or not. The sending module 12 can be a wireless communication chip that can receive wireless signals, including the global system for mobile communication (GSM), code division multiple access (CDMA), DCS, personal communication services (PCS), 3G, or wireless LAN (WLAN). The sending module 12 is used for providing portable communication to contact a relative or an organization instantaneously for saving the life of the user.

Please refer to FIGS. 1 and 4. The vital sign sending apparatus includes a sending module 12 that transmits information via wireless communication technology, a positioning module 14 (as shown in FIG. 6) that is linked with the sending module 2 and can receive satellite signals from a satellite positioning system 2, and a sensing module 16 that is linked with the sending module 12 and contacts the user's body for monitoring the vital signs of the user. The sending module 12 includes a wireless communication unit 120 for providing the wireless communication technology, a software unit 122 electrically linked with the wireless communication unit 120 for judging whether the vital signs of the user are abnormal or not, a battery unit 124 electrically linked with the wireless communication unit 120 and providing power, and a receiver/amplifier unit 126 electrically linked with the wireless communication unit 120 for providing the sound input and output functions. The positioning module 14 includes a positioning system for receiving satellite signals, and a micro processing unit 140. The sending module 12, the positioning module 14 and the sensing module 16 are combined and installed into a device, such a wrist type vital sign sending apparatus, as shown in FIG. 5. By measuring the pulse and blood pressure of the user 5, the condition of the user is monitored. Alternatively, the sending module 12, the positioning module 14 and the sensing module 16 can be installed in different devices. For example, the sending module 12 and the positioning module 14 can be installed in a cell phone if the user carries the cell phone. The sensing module 16 can be a wrist type sensing module for measuring the user's pulse or blood pressure. Alternatively, the sensing module 16 can be combined with a heart rhythm regulator installed in the body. The sensing module 16 can further be a brainwaves monitor, or a heartbeat monitor for obtaining precise measurements. For example, a quantum interference unit is made of superconductor for detecting changes in magnetic fields produced by a user's brainwaves or heartbeat.

When the sending module 12, the positioning module 14 and the sensing module 16 are installed in different devices, the vital sign sending apparatus 1 further includes a memory unit 130 for recording vital signs. The memory unit 130 can be combined with the sensing module 16 or the wireless communication unit 120. The vital signs recorded by the memory unit 130 are sent to the target person via the wireless communication unit 120.

When the sending module 12, the positioning module 14 and the sensing module 16 are installed in different devices, the vital sign sending apparatus 1 further includes a second wireless communication unit 132. The second wireless communication unit 132 can link with the sensing module 16 and the wireless communication unit 120 via short distance wireless communication technology. The short distance wireless communication technology includes active RFID, DSRC, Bluetooth, WiFi, NFC, or ZigBee etc.

The vital sign sending method and a sending apparatus thereof of present invention can monitor the conditions of a user's body, and store information regarding the user's vital sign into the vital sign sending apparatus 1 or transmit the vital signs to an external server. The present invention forcibly generates a sending function to inform a target person 3 under special or urgent conditions to achieve the emergency lifesaving function.

The description above only illustrates specific embodiments and examples of the invention. The invention should therefore cover various modifications and variations made to the herein-described structure and operations of the invention, provided they fall within the scope of the invention as defined in the following appended claims.

What is claimed is:

1. A vital sign sending method, the step comprising:
wearing a vital sign sending apparatus by a user; wherein the vital sign sending apparatus includes a positioning module fitted with a satellite positioning system, a sending module that can transmit information via a wireless communication system, and a sensing module that contacts the user's body and monitors the vital signs of the user;
obtaining vital signs detected by the vital sign sending apparatus over a fixed period, and judging whether the vital signs of the user are abnormal or not via a software;
when the vital sign of the users are abnormal, the vital sign sending apparatus forcibly generates a sending function to inform a target person; and
the location and the vital signs are transmitted to the target person.

2. The vital sign sending method as claimed in claim 1, wherein the step of a user wearing a vital sign sending apparatus further comprises setting a set of emergency telephone numbers or accounts into the vital sign sending apparatus 1 in advance.

3. The vital sign sending method as claimed in claim 2, wherein the step of the vital sign sending apparatus forcibly generating a sending function further comprises automatically contacting the emergency telephone numbers or accounts.

4. The vital sign sending method as claimed in claim 3, wherein the emergency telephone numbers or accounts are arranged in order of importance in advance, and the order is used as an automatic contacting sequence.

5. The vital sign sending method as claimed in claim 3, wherein the contacting method includes making a call, sending a short message, or transmitting an instantaneous electronic information.

6. The vital sign sending method as claimed in claim 5, wherein the step of transmitting information regarding the user's location and vital signs to the target person further comprises sending a short message with the user's location and vital signs, via instantaneous e-mail to the target person.

7. The vital sign sending method as claimed in claim 6, wherein the step of the vital sign sending apparatus forcibly generating a sending function further comprises:
the target person calling the user, and monitoring the environmental condition of the user via a voice system or an Internet interface.

8. The vital sign sending method as claimed in claim 5, wherein the step of transmitting information regarding the user's location and vital signs to the target person further comprises:
making a call to the target person; and
obtaining the location information and vital signs of the user by the target person via a voice system.

9. The vital sign sending method as claimed in claim 8, wherein the step of transmitting information regarding the user's location and vital signs to the target person further comprises:
monitoring by the target person of the environmental conditions of the user via a voice system or an Internet interface.

10. The vital sign sending method as claimed in claim 1, wherein the target person is a person, a private organization or a governmental organization.

11. The vital sign sending method as claimed in claim 10, wherein the target person is a relative, a hospital, a police station, or an emergency organization.

12. The vital sign sending method as claimed in claim 1, wherein the step of the vital sign sending apparatus forcibly generating a sending function further comprises:
the target person selects to monitor the environmental conditions of the user.

13. The vital sign sending method as claimed in claim 1, wherein the step of transmitting information regarding the user's location and vital signs to the target person further comprises:
actively transmitting information regarding the user's location and vital signs to the target person, or passively transmitting information regarding the user's location and vital signs to the target person.

14. The vital sign sending method as claimed in claim 1, wherein the satellite positioning system is a global positioning system (GPS), a global orbiting navigation satellite system (GLONASS), a Dipper positioning system, or a Galileo commercial satellite positioning system.

15. The vital sign sending method as claimed in claim 1, wherein the sending module is a global system for mobile communication (GSM), a code division multiple access (CDMA), a DCS, a personal communication services (PCS), a 3G, or a wireless LAN (WLAN).

16. The vital sign sending method as claimed in claim 1, wherein the sensing module uses brainwave, pulse, heartbeat or sweat gland monitoring technology.

17. A vital sign sending apparatus, comprising:
a sending module that transmits information via wireless communication technology;
a positioning module that is linked with the sending module and receives satellite signals from a satellite positioning system; and
a sensing module that is linked with the sending module and contacts the user's body for monitoring the vital signs of the user;
wherein the sending module includes a wireless communication unit for providing wireless communication technology, a software unit electrically linked with the wireless communication unit for judging whether the vital signs of the user are abnormal or not, a battery unit electrically linked with the wireless communication unit for providing power, and a receiver/amplifier unit electrically linked with the wireless communication unit for providing the sound input and output functions;
wherein the positioning module includes a positioning system for receiving satellite signals, and a micro processing unit.

18. The vital sign sending apparatus as claimed in claim 17, wherein the sending module and the positioning module are installed inside a cell phone.

19. The vital sign sending apparatus as claimed in claim 17, further comprising a memory unit for recording the user's vital sign.

20. The vital sign sending apparatus as claimed in claim 17, wherein the sensing module is used for monitoring the user's brainwaves, pulse, heartbeat or sweat glands.

21. The vital sign sending apparatus as claimed in claim 20, wherein the sensing module is a heart rhythm regulator.

22. The vital sign sending apparatus as claimed in claim 20, wherein the sensing module includes a quantum interference unit made of superconductor for detecting changes in a magnetic field produced by the user's brainwaves or heartbeat.

23. The vital sign sending apparatus as claimed in claim 17, wherein the sending module, the positioning module and the sensing module are installed in a device.

24. The vital sign sending apparatus as claimed in claim 17, further comprising a second wireless communication unit, wherein the second wireless communication unit is linked with the sensing module and the wireless communication unit by a short distance wireless communication technology.

25. The vital sign sending apparatus as claimed in claim 24, wherein the short distance wireless communication technology is active RFID, DSRC, Bluetooth, WiFi, NFC, or ZigBee.

26. The vital sign sending apparatus as claimed in claim 17, wherein the positioning module is a global positioning system (GPS), a global orbiting navigation satellite system (GLONASS), a Dipper positioning system, or a Galileo commercial satellite positioning system.

27. The vital sign sending apparatus as claimed in claim 17, wherein the wireless communication unit is a wireless communication chip including a global system for mobile communication (GSM), a code division multiple access (CDMA), a DCS, a personal communication services (PCS), a 3G, or a wireless LAN (WLAN).
